(19) United States
(12) Patent Application Publication
(10) Pub. No.: US 2007/0288042 A1
(43) Pub. Date: Dec. 13, 2007

(54) Flexible debridement device

(76) Inventors: Jon C. Serbousek, Memphis, TN (US); Carlos E. Gil, Collierville, TN (US)

Correspondence Address: LARSON NEWMAN ABEL POLANSKY & WHITE, LLP, 5914 WEST COURTYARD DRIVE, SUITE 200, AUSTIN, TX, 78730, US

(21) Appl. No.: 11/448,556

(22) Filed: Jun. 7, 2006

(51) Int. Cl. A61B 17/22

(52) U.S. Cl. 606/160

(57) ABSTRACT

A flexible debridement device is disclosed and can include a shaft having a proximal end and a distal end. A debridement tool can be coupled to the distal end of the shaft. The flexible debridement device can be moved between a straight position and a plurality of bent positions. Additionally, the flexible debridement device can automatically return substantially to the straight position after a bending force is removed from the flexible debridement device.

### FIELD OF THE DISCLOSURE

The present disclosure relates generally to surgical tools. More specifically, the present disclosure relates to surgical tools used to remove osteolytic lesions.

### BACKGROUND

Osteolytic lesions can be found on arthroplasty implants after several years of implantation. Revision surgery can be used to treat patients with osteolytic lesions. The revision surgery can include removing the implant and replacing the implant with a new implant. Alternatively, the revision surgery can include removing and replacing a liner made from ultra-high molecular weight polyethylene (UHMWP). In either case, it may be desirable to remove the osteolytic lesions from around the implant.

Currently, surgical curettes made from stainless steel can be used to remove osteolytic lesions. A surgical curette can include a stainless steel shaft that can be bent intraoperatively to a desired shape with a bending tool. After the surgery, the bent surgical curette is discarded. Accordingly, there is a need for a surgical curette that can be bent during surgery to a plurality of positions and returned to a straight position for re-use.

### BRIEF DESCRIPTION OF THE DRAWINGS

FIG. 1 is a first plan view of a first embodiment of a flexible debridement device;

FIG. 2 is a second plan view of the first embodiment of the flexible debridement device;

FIG. 3 is a cross-section view of the first embodiment of the flexible debridement device taken along line 3-3 in FIG. 1;

FIG. 4 is a first detail view of the first embodiment of the flexible debridement device taken at circle 4 in FIG. 1;

FIG. 5 is a second detail view of the first embodiment of the flexible debridement device taken at circle 5 in FIG. 2;

FIG. 6 is a cross-section view of the first embodiment of the flexible debridement device taken at line 6-6 in FIG. 4;

FIG. 7 is a plan view of the first embodiment of the flexible debridement device bent to a plurality of positions;

FIG. 8 is a plan view of the first embodiment of the flexible debridement device bent to a maximum bent position;

FIG. 9 is a first plan view of a second embodiment of a flexible debridement device;

FIG. 10 is a second plan view of the second embodiment of the flexible debridement device;

FIG. 11 is a cross-section view of the second embodiment of the flexible debridement device taken along line 11-11 in FIG. 9;

FIG. 12 is a first detail view of the second embodiment of the flexible debridement device taken at circle 12 in FIG. 9;

FIG. 13 is a second detail view of the second embodiment of the flexible debridement device taken at circle 13 in FIG. 10;

FIG. 14 is a cross-section view of the second embodiment of the flexible debridement device taken at line 14-14 in FIG. 12;

FIG. 15 is a plan view of the second embodiment of the flexible debridement device bent to a plurality of positions;

FIG. 16 is a plan view of the second embodiment of the flexible debridement device bent to an extreme position; and

FIG. 17 is a method of revising an arthroplasty implant surgery.

### DETAILED DESCRIPTION OF THE DRAWINGS

A flexible debridement device is disclosed and can include a shaft having a proximal end and a distal end. A debridement tool can be coupled to the distal end of the shaft. The flexible debridement device can be moved between a straight position and a plurality of bent positions. Additionally, the flexible debridement device can automatically return substantially to the straight position after a bending force is removed from the flexible debridement device.

In another embodiment, a flexible debridement device is disclosed and can include a shaft having a proximal end and a distal end. A handle can be attached to the proximal end of the shaft. Also, a debridement tool can be attached to the distal end of the shaft. The flexible debridement device can be moved between a straight position in which the curette is collinear with the handle and a plurality of bent positions in which the curette is non-collinear with the handle. Further, the flexible debridement device can automatically return substantially to the straight position when a bending force is removed from the flexible debridement device.

In yet another embodiment, a method of revising an arthroplasty implant is disclosed and can include exposing the arthroplasty implant and inserting a flexible debridement device to an area around the arthroplasty implant. Moreover, the method can include applying a bending force to the flexible debridement device to bend the flexible debridement device from a straight position to one of a plurality of bent positions. The method can also include removing an osteolytic lesion from the area around the arthroplasty implant.

In another embodiment, a debridement device is disclosed and can include a shaft having a proximal end and a distal end. A debridement tool can be coupled to the distal end of the shaft. The debridement device can be moved between a straight position and a plurality of bent positions. Further, the debridement device can automatically return substantially to the straight position when exposed to heat above a transformation temperature.

In still another embodiment, a method of treating a patient is disclosed and can include exposing a musculoskeletal lesion. The method can also include bending a debridement device from a substantially straight position to one of a plurality of bent positions and inserting an end of the debridement device into the patient. Further, the method can include removing the musculoskeletal lesion using the debridement device.

### DESCRIPTION OF A FIRST EMBODIMENT OF A FLEXIBLE DEBRIDEMENT DEVICE

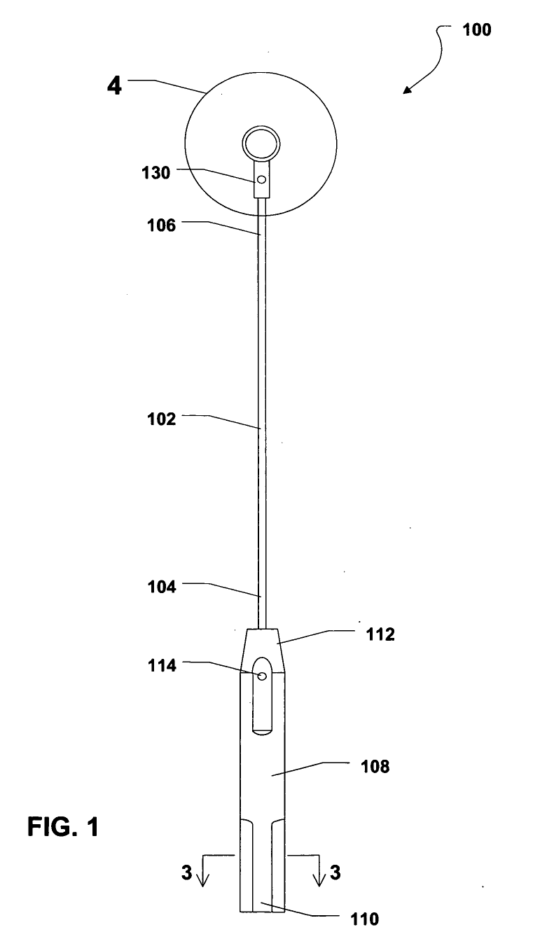

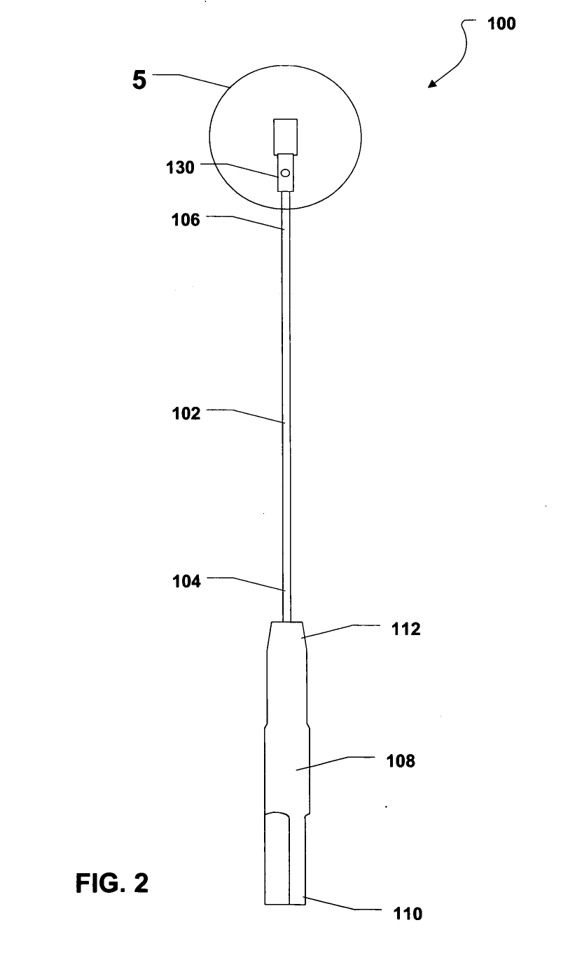

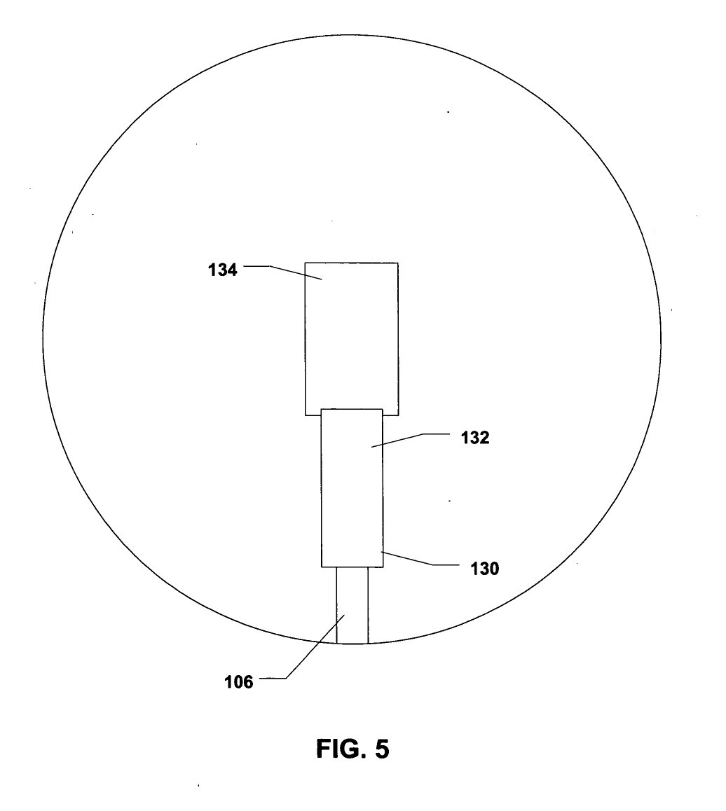

Referring to FIG. 1 through FIG. 8, a first embodiment of a flexible debridement device is shown and is generally designated 100. As shown in FIG. 1 and FIG. 2, the flexible debridement device 100 can include a shaft 102 that can include a proximal end 104 and a distal end 106.

FIG. 1 and FIG. 2 further indicate that the flexible debridement device 100 can include a handle 108 attached or otherwise affixed to the shaft 102. In a particular embodiment, the handle 108 can be made from one or more rigid materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. Alternatively, the handle 108 can be made from any other substantially rigid materials.

The handle 108 can include a proximal end 110 and a distal end 112. Further, the distal end 112 of the handle 108 can be attached to the proximal end 104 of the shaft 102. In a particular embodiment, the proximal end 104 of the shaft 102 can be installed within the distal end 112 of the handle 108. Moreover, a pin 114 or other suitable fastener can be inserted through the distal end 112 of the handle 108 and the proximal end 104 of the shaft 102. The pin 114 can secure the proximal end 104 of the shaft 102 within the distal end 112 of the handle 108.

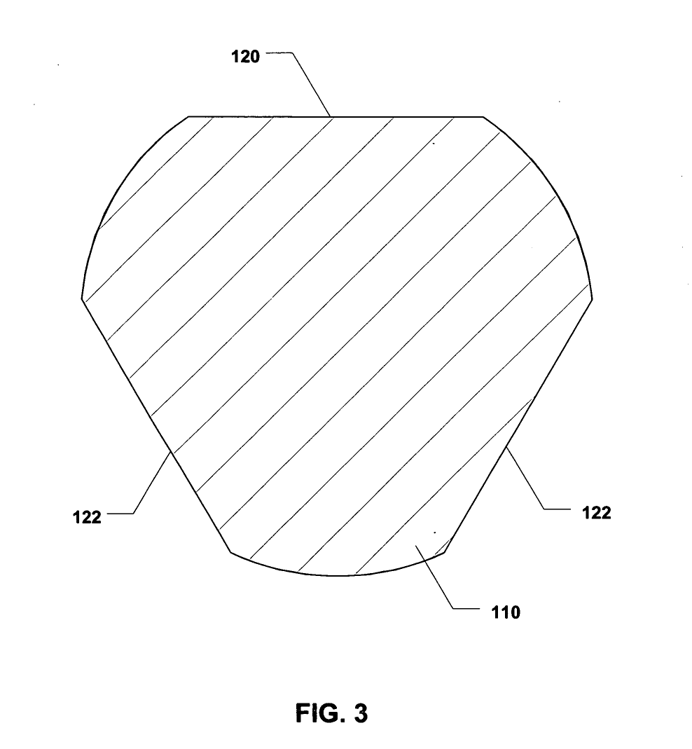

Referring to FIG. 3, a cross-section of the proximal end 110 of the handle 108 is shown. In a particular embodiment, the proximal end 110 of the handle 108 can be configured to be received in a drill, e.g., a surgical drill. Specifically, the proximal end 110 of the handle 108 can include a first flat surface 120, a second flat surface 122, and a third flat surface 124. In particular embodiment, the first flat surface120 can be arranged at a sixty degree (60°) angle with respect to the second flat surface 122. The second flat surface 122 can be arranged at a sixty degree (60°) angle with respect to the third flat surface 124. Also, the third flat surface 124 can be arranged at a sixty degree (60°) angle with respect to the first flat surface 120. Accordingly, the flat surfaces 120, 122, 124 can be equally spaced radially around the proximal end 110 of the handle 108.

As illustrated in FIG. 1 and FIG. 2, the flexible debridement device 100 can also include a debridement tool, e.g., a curette 130 that can be affixed to the distal end 106 of the shaft 102. In a particular embodiment, the curette 130 can be made from one or more rigid materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. Alternatively, the curette 130 can be made from any other substantially rigid materials.

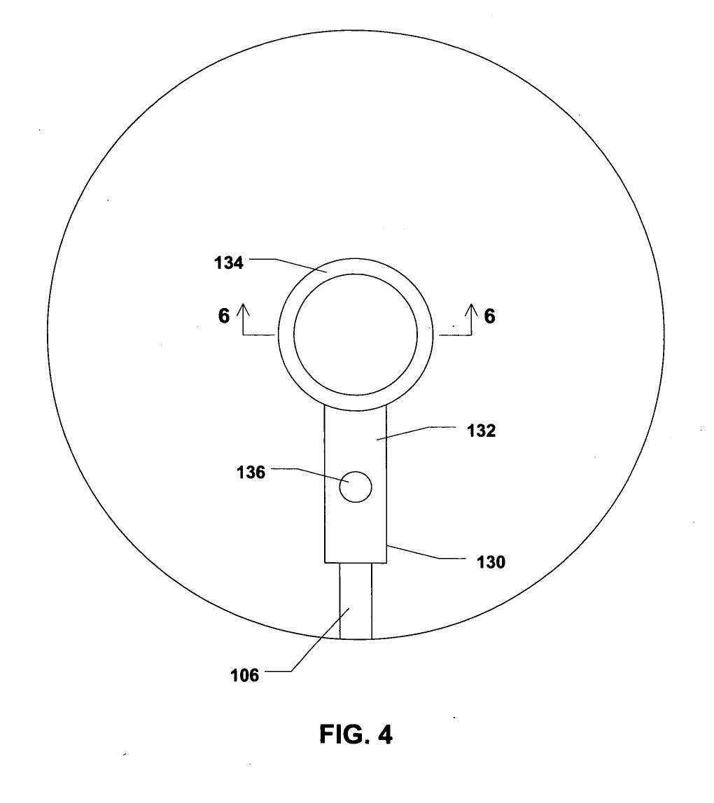

FIG. 4 through FIG. 6 depict details of the curette 130. As shown, the curette 130 can include a collar 132. A ring 134 can extend from the collar 132. In a particular embodiment, the collar 132 can be inserted over the distal end 106 of the shaft 102. Moreover, a pin 136 or other suitable fastener can be inserted through the collar 132 and the distal end 106 of the shaft 102 and the pin 136 can secure the curette 130 to the distal end 106 of the shaft 102.

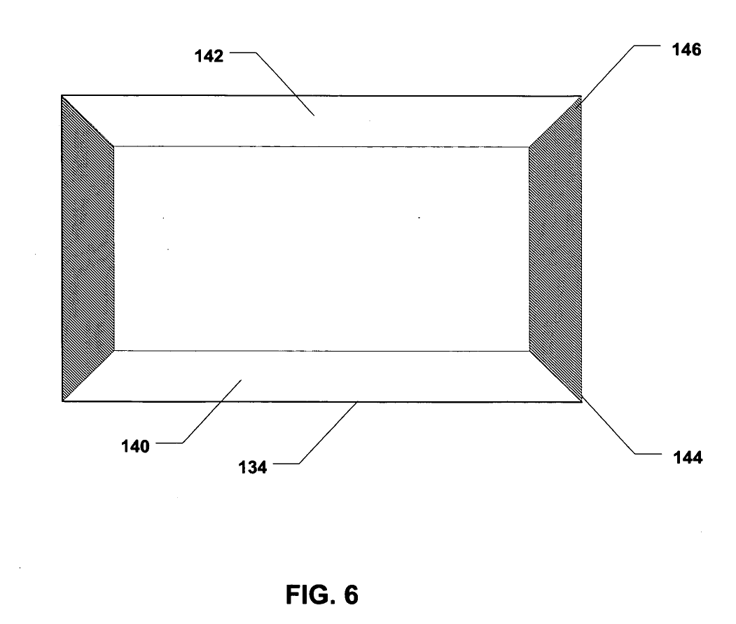

FIG. 6 depicts the ring 134 of the curette 130 in cross-section. As shown, the ring 134 includes a first open end 140 and a second open end 142. In a particular embodiment, the first open end 140 of the ring 134 of the curette 130 can be angled, or otherwise shaped, to establish a first cutting edge 144 and the second open end 142 of the ring 134 of the curette 130 can be angled, or otherwise shaped, to establish a second cutting edge 146. During use, the cutting edges 144, 146 can be used to scrape, cut, shave, or otherwise remove osteolytic lesions formed around an implant, e.g., a hip implant, a spinal implant, or other implant.

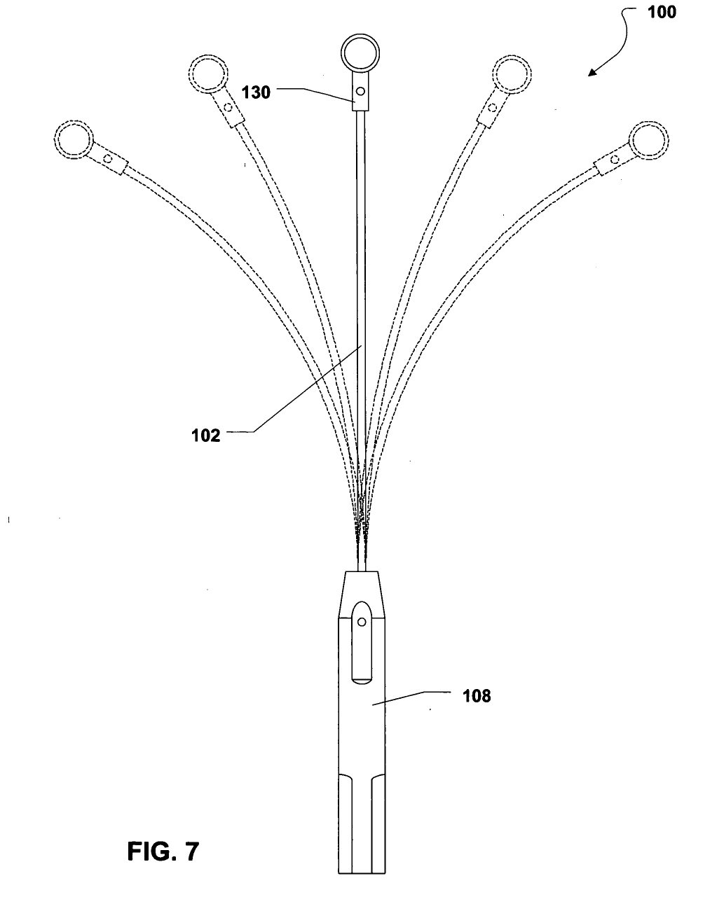

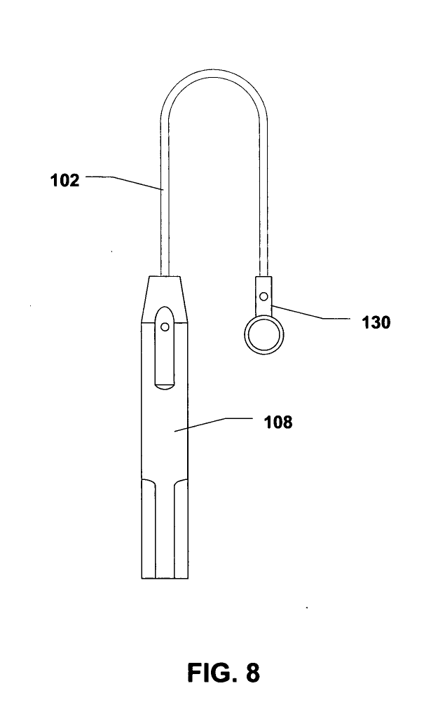

FIG. 7 and FIG. 8 indicate that the flexible debridement device 100, e.g., the shaft 102, can be bent from a straight position to a plurality of curved, or bent, positions. FIG. 8 shows that the flexible debridement device 100 can be bent from a straight position in which the curette 130 is substantially collinear, or aligned, with the handle 108 to a maximum bent position in which the curette 130 is parallel to the handle 108. Accordingly, the flexible debridement device 100 can be bent so that the curette 130 is rotated at least one hundred and eighty degrees (180°). Further, after a bending force, which is used to bend, or otherwise flex, the flexible debridement device 100, is removed from the flexible debridement device 100, the elasticity, or flexibility, of the shaft 102 can return the flexible debridement device 100 substantially to the straight position.

In a particular embodiment, the shaft 102 can be made from an elastic, or flexible, material. For example, the shaft 102 can be made from a metal alloy material, a polymer material, a composite material, or a combination thereof. For example, the metal alloy material can be a nickel titanium alloy, such as nitinol. The polymer material can be an elastomer, such as polyurethane. Also, the composite material can be carbon fiber. The shaft 102 can also be made from a multi-filament cable or any other flexible material.

In another embodiment, the shaft 102 can be made from a shape memory material. The shape memory material can be a shape memory metal, a shape memory polymer, or a combination thereof. For example, the shape memory metal can be a nickel titanium alloy, such as nitinol. In such an embodiment, the shaft 102 can be bent to a particular shape and used as described herein. Thereafter, the shaft 102 can be returned to a substantially straight shape by exposing the shaft 102 to heat. For example, the shaft 102 can be dipped, or otherwise bathed, in heated saline in order to return the shaft 102 to a substantially straight shape. In a particular embodiment, the saline can be heated to a temperature above a transformation temperature in order to return the shaft 102 to a substantially straight shape.

Further, in additional embodiments, other debridement tools may be attached, or otherwise affixed, to the distal end 106 of the shaft 102. For example, a knife, a scraper, a brush, an ultra-sound probe, a radio frequency probe, an aspiration tool, or some other debridement tool may be attached to the distal end 106 of the shaft 102.

### DESCRIPTION OF A SECOND EMBODIMENT OF A FLEXIBLE DEBRIDEMENT DEVICE

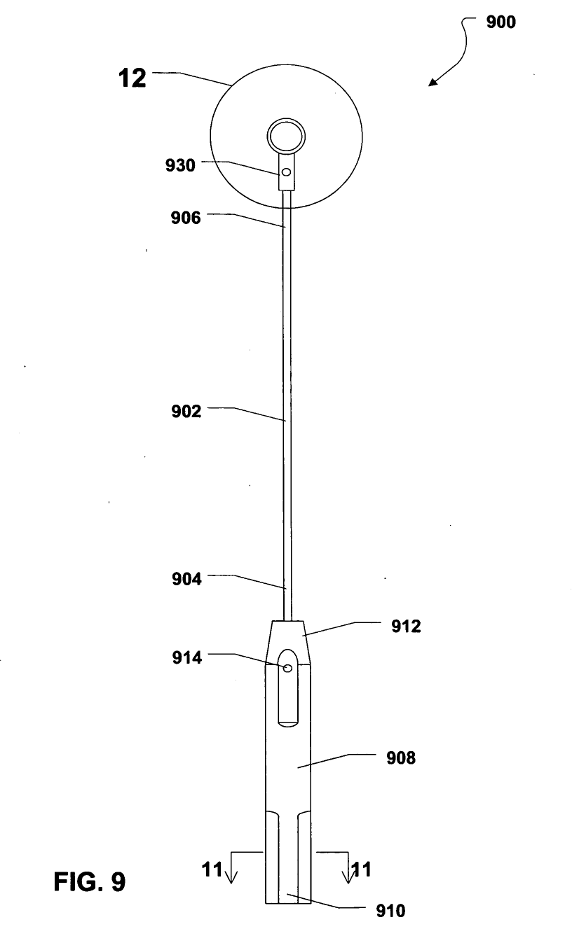

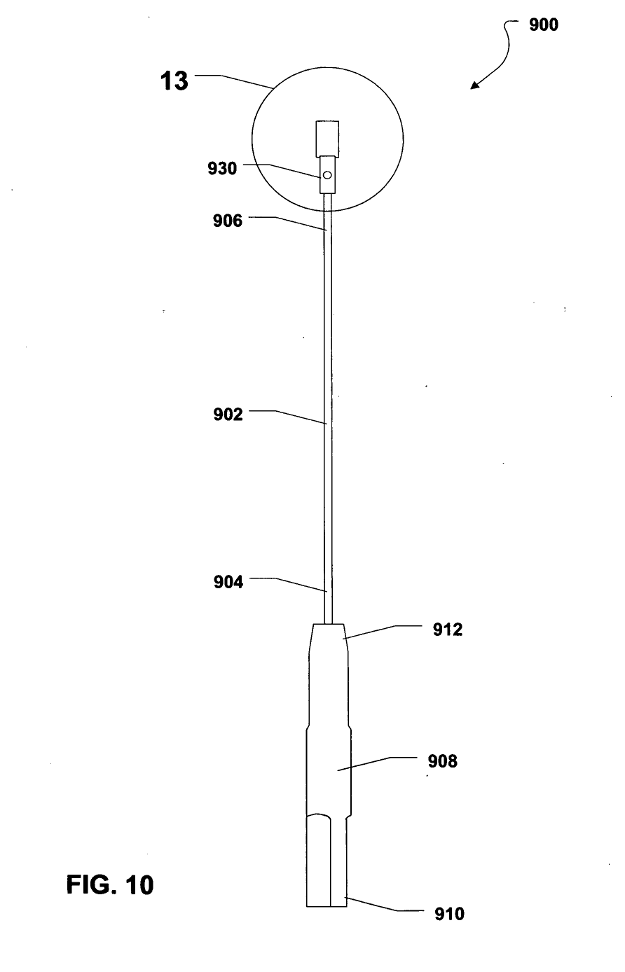

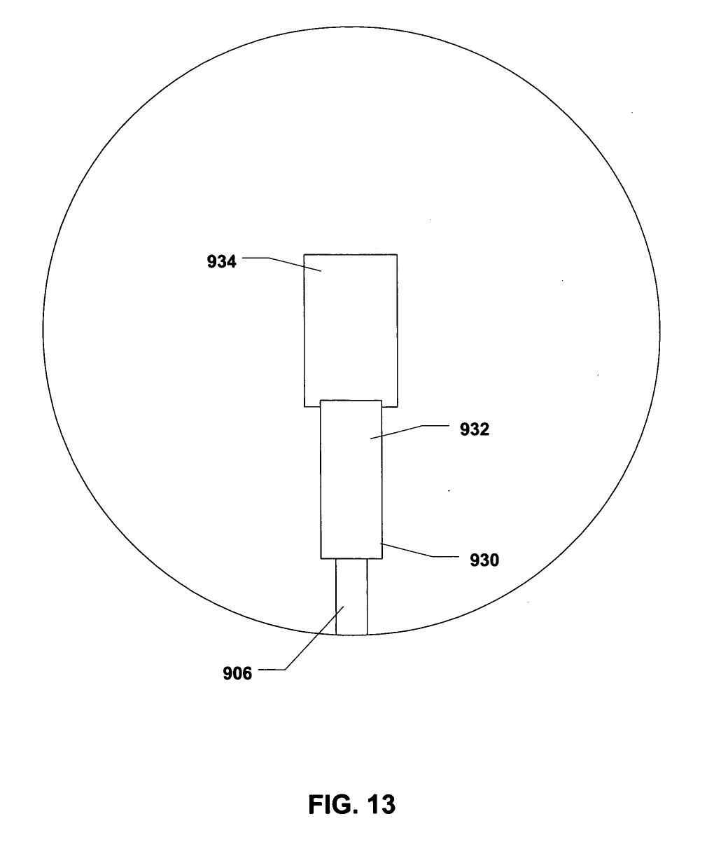

Referring to FIG. 9 through FIG. 16, a second embodiment of a flexible debridement device is shown and is generally designated 900. As shown in FIG. 9 and FIG. 10, the flexible debridement device 900 can include a shaft 902 that can include a proximal end 904 and a distal end 906.

FIG. 9 and FIG. 10 further indicate that the flexible debridement device 900 can include a handle 908 attached or otherwise affixed to the shaft 902. In a particular embodiment, the handle 908 can be made from one or more rigid materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. Alternatively, the handle 908 can be made from any other substantially rigid materials.

The handle 908 can include a proximal end 910 and a distal end 912. Further, the distal end 912 of the handle 908 can be attached to the proximal end 904 of the shaft 902. In a particular embodiment, the proximal end 904 of the shaft 902 can be installed within the distal end 912 of the handle 908. Moreover, a pin 914 or other suitable fastener can be inserted through the distal end 912 of the handle 908 and the proximal end 904 of the shaft 902. The pin 914 can secure the proximal end 904 of the shaft 902 within the distal end 912 of the handle 908.

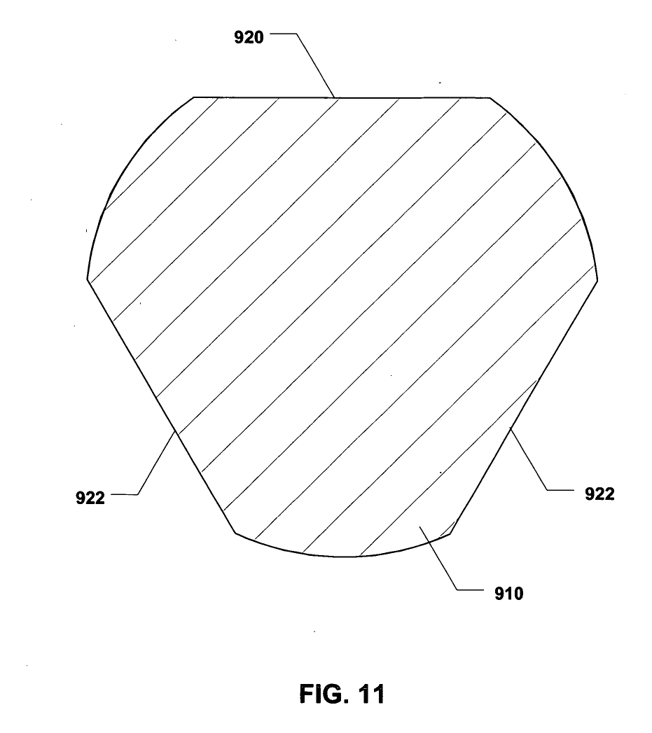

Referring to FIG. 11, a cross-section of the proximal end 910 of the handle 908 is shown. In a particular embodiment, the proximal end 910 of the handle 908 can be configured to be received in a drill, e.g., a surgical drill. Specifically, the proximal end 910 of the handle 908 can include a first flat surface 920, a second flat surface 922, and a third flat surface 924. In particular embodiment, the first flat surface120 can be arranged at a sixty degree (60°) angle with respect to the second flat surface 922. The second flat surface 922 can be arranged at a sixty degree (60°) angle with respect to the third flat surface 924. Also, the third flat surface 924 can be arranged at a sixty degree (60°) angle with respect to the first flat surface 920. Accordingly, the flat surfaces 920, 922, 924 can be equally spaced radially around the proximal end 910 of the handle 908.

As illustrated in FIG. 9 and FIG. 10, the flexible debridement device 900 can also include a debridement tool, e.g., a curette 930 that can be affixed to the distal end 906 of the shaft 902. In a particular embodiment, the curette 930 can be made from one or more rigid materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. Alternatively, the curette 930 can be made from any other substantially rigid materials.

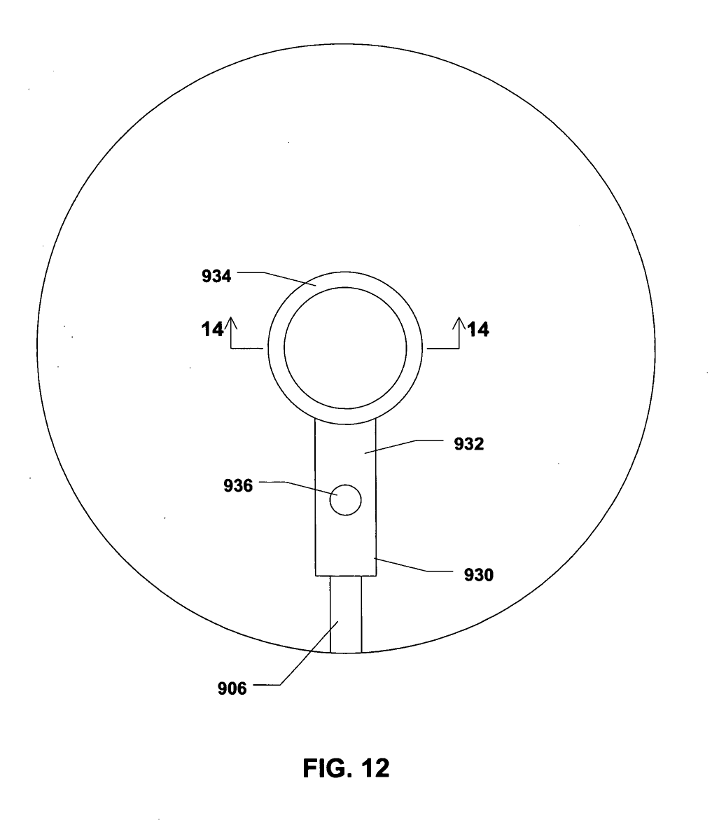

FIG. 12 through FIG. 14 depict details of the curette 930. As shown, the curette 930 can include a collar 932. A ring 934 can extend from the collar 932. In a particular embodiment, the collar 932 can be inserted over the distal end 906 of the shaft 902. Moreover, a pin 936 or other suitable fastener can be inserted through the collar 932 and the distal end 906 of the shaft 902 and the pin 936 can secure the curette 930 to the distal end 906 of the shaft 902.

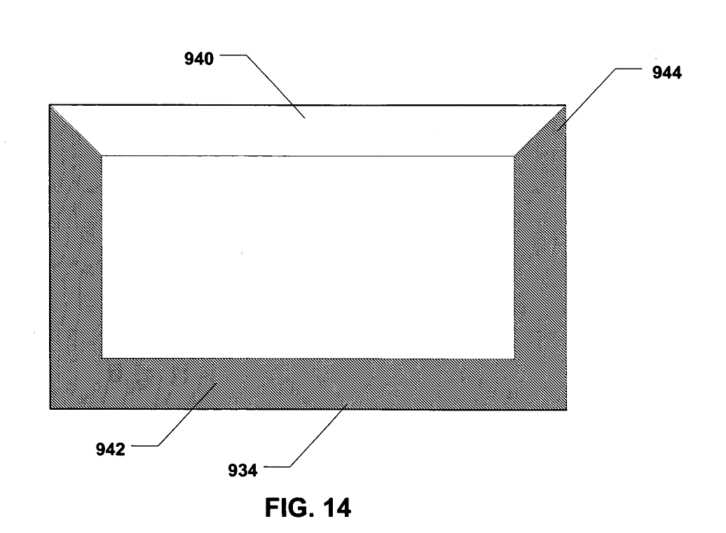

FIG. 14 depicts the ring 934 of the curette 930 in cross-section. As shown, the ring 934 includes an open end 940 and a closed end 942. In a particular embodiment, the open end 940 of the ring 934 of the curette 930 can be angled, or otherwise shaped, to establish a cutting edge 944. During use, the cutting edge 944 can be used to scrape, cut, shave, or otherwise remove osteolytic lesions formed around an implant, e.g., a hip implant, a spinal implant, or other implant.

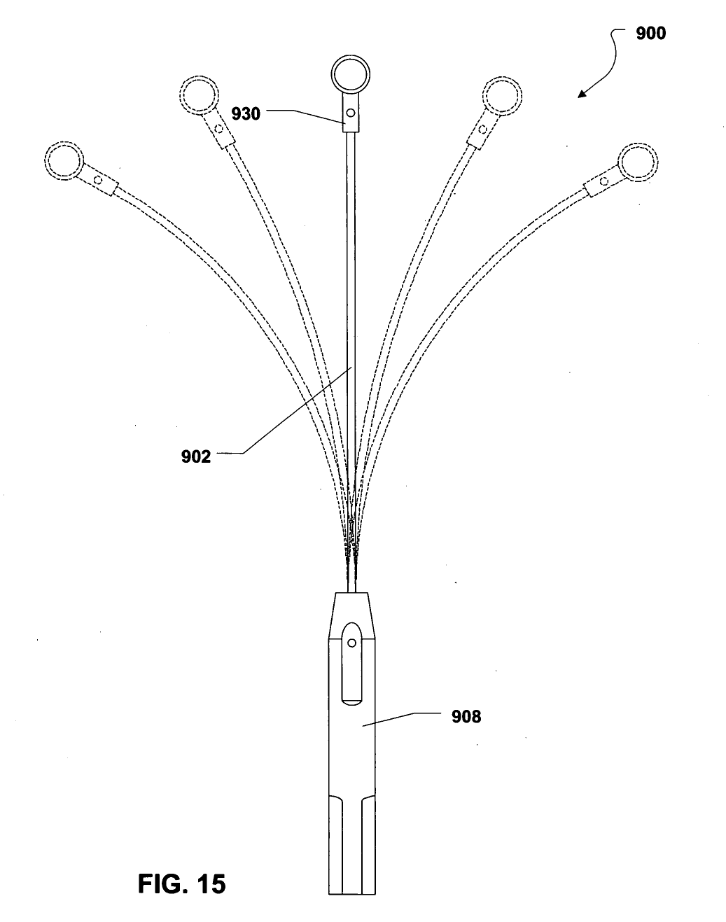

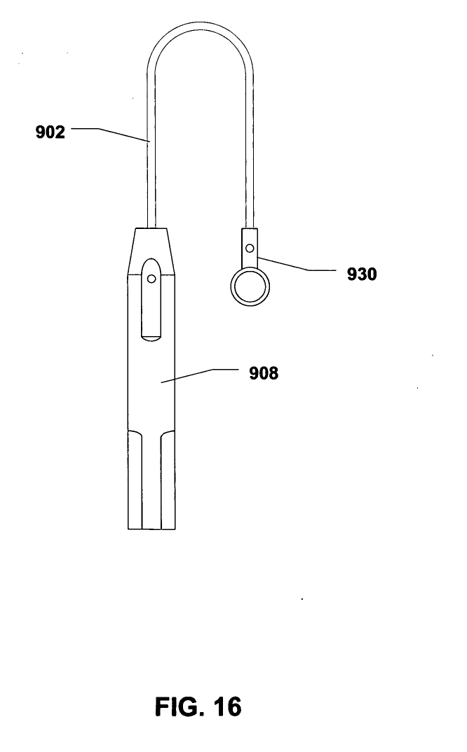

FIG. 15 and FIG. 16 indicate that the flexible debridement device 900, e.g., the shaft 902, can be bent from a straight position to a plurality of curved, or bent, positions. FIG. 16 shows that the flexible debridement device 900 can be bent from a straight position in which the curette 930 is substantially collinear, or aligned, with the handle 908 to a maximum bent position in which the curette 930 is parallel to the handle 908. Accordingly, the flexible debridement device 900 can be bent so that the curette 930 is rotated at least one hundred and eighty degrees (180°). Further, after a bending force, which is used to bend, or otherwise flex, the flexible debridement device 900, is removed from the flexible debridement device 900, the elasticity, or flexibility, of the shaft 902 can return the flexible debridement device 900 substantially to the straight position.

In a particular embodiment, the shaft 902 can be made from an elastic, or flexible, material. For example, the shaft 902 can be made from a metal alloy material, a polymer material, a composite material, or a combination thereof. For example, the metal alloy material can be a nickel titanium alloy, such as nitinol. The polymer material can be an elastomer, such as polyurethane. Also, the composite material can be carbon fiber. The shaft 902 can also be made from a multi-filament cable or any other flexible material.

In another embodiment, the shaft 902 can be made from a shape memory material. The shape memory material can be a shape memory metal, a shape memory polymer, or a combination thereof. For example, the shape memory metal can be a nickel titanium alloy, such as nitinol. In such an embodiment, the shaft 902 can be bent to a particular shape and used as described herein. Thereafter, the shaft 902 can be returned to a substantially straight shape by exposing the shaft 902 to heat. For example, the shaft 902 can be dipped, or otherwise bathed, in heated saline in order to return the shaft 902 to a substantially straight shape. In a particular embodiment, the saline can be heated to a temperature above a transformation temperature in order to return the shaft 902 to a substantially straight shape.

Further, in additional embodiments, other debridement tools may be attached, or otherwise affixed, to the distal end 906 of the shaft 902. For example, a knife, a scraper, a brush, an ultra-sound probe, a radio frequency probe, an aspiration tool, or some other debridement tool may be attached to the distal end 906 of the shaft 902.

### Description of a Method of Revising an Arthroplasty Implant Surgery

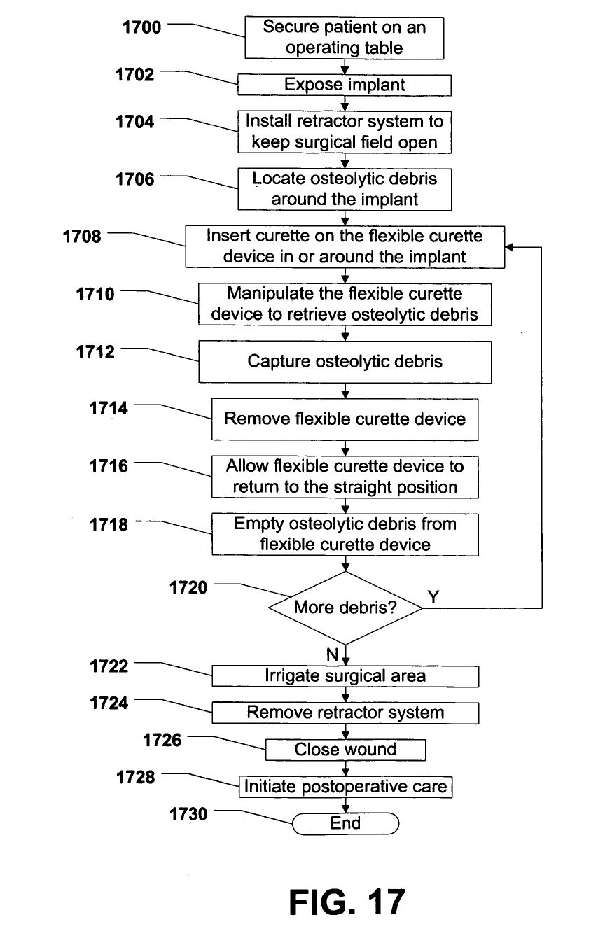

Referring now to FIG. 17, a method of revising an arthroplasty implant surgery is shown and commences at block 1700. The present method can be used to revise a spinal implant, a hip implant, a knee implant, or any other implant in which osteolytic lesions can be problematic. At block 1700, the patient can be secured on an operating table. Depending on the implant to be revised, the patient can be secured in a prone position, a supine position, a lateral decubitus position, or another position well known in the art.

Moving to block 1702, an implant can be exposed. At block 1704, a retractor system can be installed in order to keep the surgical field open. Further, at block 1706 osteolytic debris, or lesions, can be located around the implant. At block 1708, a curette on a flexible debridement device can be inserted in or around the implant. In a particular embodiment, the flexible debridement device can be a flexible debridement device according to one or more embodiments described herein.

At block 1710, the flexible debridement device can be manipulated in order to retrieve the osteolytic debris. In a particular embodiment, a bending force can be applied to the flexible debridement device and the flexible debridement device can be bent, or otherwise flexed, in order to retrieve the osteolytic debris. Further, the flexible debridement device can be used to scrape, cut, or otherwise remove osteolytic lesions formed around or near the implant.

Proceeding to block 1712, the osteolytic debris can be captured. In a particular embodiment, the osteolytic debris can be captured using the flexible debridement device. Alternatively, the osteolytic debris can be captured using a pair of forceps. Continuing to block 1714, the flexible debridement device can be removed from the surgical field. At block 1716, the flexible debridement device can be allowed to return to the straight position. The flexible debridement device can be allowed to return to the straight position by removing the bending force from the flexible debridement device. Moreover, at block 1718, osteolytic debris can be emptied from the flexible debridement device.

Moving to decision step 720, it can be determined whether more osteolytic debris, or lesions, exists around the implant. If so, the method can return to block 1708 and continue as described herein. On the other hand, the method can move to block 1722 and the surgical area can be irrigated. At block 1724, the retractor system can be removed. Further, at block 1726, the surgical wound can be closed. The surgical wound can be closed by simply allowing the patient's skin to close due to the elasticity of the skin. Alternatively, the surgical wound can be closed using sutures, surgical staples, or any other suitable surgical technique well known in the art. At block 1728, postoperative care can be initiated. The method can end at state 1730.

In an alternative embodiment, the flexible debridement device can be used to remove other musculoskeletal lesions. The musculoskeletal lesion can be exposed and the flexible debridement tool can be bent or flexed in order to facilitate removal of the musculoskeletal lesion.

### CONCLUSION

With the configuration of structure described above, the flexible debridement device provides a device that can be used to remove osteolytic lesions or debris from around an arthroplasty implant. The flexible debridement device can be bent, or otherwise manipulated, to remove and retrieve and osteolytic lesions or debris. After use, the elasticity of the flexible debridement device can automatically return the flexible debridement device to a straight position from one of numerous bent positions. Alternatively, the flexible debridement device can be returned to a straight position by exposing the shaft of the flexible debridement device to heat at a temperature above a transformation temperature.

The flexible debridement device can be a single use device or a multiple use device. Further, the flexible debridement device can be packaged as part of a kit that can include one or more flexible debridement devices, one or more brushes, and one or more suction tips.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. A flexible debridement device, comprising:
a shaft having a proximal end and a distal end; and
a debridement tool coupled to the distal end of the shaft, wherein the flexible debridement device is movable between a straight position and a plurality of bent positions and wherein the flexible debridement device automatically returns substantially to the straight position after a bending force is removed from the flexible debridement device.

2. The flexible debridement device of claim 1, further comprising:
a handle coupled to the proximal end of the shaft opposite the debridement tool.

3. The flexible debridement device of claim 2, wherein in the straight position the handle is substantially collinear with the debridement tool.

4. The flexible debridement device of claim 4, wherein in the plurality of bent positions, the handle is non-collinear with the debridement tool.

5. The flexible debridement device of claim 4, wherein the flexible debridement device can be bent to a maximum bent position in which the debridement tool is moved at least one hundred and eighty degrees (180°) relative to the handle.

6. The flexible debridement device of claim 4, wherein the flexible debridement device can be bent such that the debridement tool is substantially parallel to the handle.

7. The flexible debridement device of claim 1, wherein the debridement tool comprises a curette, a knife, a scraper, a brush, an ultrasound probe, a radio frequency probe, an aspiration device, or a combination thereof.

8. The flexible debridement device of claim 7, wherein the curette comprises:
a collar configured to be inserted over the distal end of the shaft; and
a ring extending from the collar.

9. The flexible debridement device of claim 8, wherein the ring comprises:
a first open end; and
a second open end.

10. The flexible debridement device of claim 9, wherein the first open end includes a first cutting edge.

11. The flexible debridement device of claim 10, wherein the second open end includes a second cutting edge.

12. The flexible debridement device of claim 8, wherein the ring comprises:
an open end; and
a closed end.

13. The flexible debridement device of claim of claim 12, wherein the open end includes a cutting edge.

14. The flexible debridement device of claim 12, wherein the handle comprises a proximal end and a distal end and wherein the shaft is configured to be inserted into the distal end of the handle.

15. The flexible debridement device of claim 14, wherein the proximal end of the handle is configured to be received within a drill.

16. The flexible debridement device of claim 15, wherein the proximal end of the handle comprises:
a first flat surface;
a second flat surface oriented at a sixty degree (60°) angle with respect to the first flat surface; and
a third flat surface oriented at a sixty degree (60°) angle with respect to the second flat surface and oriented at a sixty degree (60°) angle with respect to the first flat surface.

17. The flexible debridement device of claim 1, wherein the shaft comprises a metal alloy material, a polymer material, a composite material, or a combination thereof.

18. The flexible debridement device of claim 17, wherein the metal alloy material comprises a nickel titanium alloy.

19. The flexible debridement device of claim 17, wherein the polymer material comprises polyurethane.

20. The flexible debridement device of claim 17, wherein the composite material comprises carbon fiber.

21. The flexible debridement device of claim 1, wherein the shaft comprises a multi-filament cable.

22. A flexible debridement device, comprising:
a shaft having a proximal end and a distal end;
a handle attached to the proximal end of the shaft; and
a debridement tool attached to the distal end of the shaft, wherein the flexible debridement device is movable between a straight position in which the debridement tool is collinear with the handle and a plurality of bent positions in which the debridement tool is non-collinear with the handle and wherein the flexible debridement device automatically returns substantially to the straight position when a bending force is removed from the flexible debridement device.

23-33. (canceled)

34. A method of revising an arthroplasty implant, comprising:
exposing the arthroplasty implant;
inserting a flexible debridement device to an area around the arthroplasty implant;
applying a bending force to the flexible debridement device to bend the flexible debridement device from a straight position to one of a plurality of bent positions; and
removing an osteolytic lesion from the area around the arthroplasty implant.

35. The method of claim 34, further comprising:
capturing osteolytic debris from the area around the arthroplasty implant.

36. The method of claim 34, further comprising:
removing the flexible debridement device.

37. The method of claim 35, further comprising:
removing the bending force from the flexible debridement device to allow the flexible debridement device to return to the straight position.

38. The method of claim 37, further comprising:
emptying osteolytic debris from the flexible debridement device.

39. A debridement device, comprising:
a shaft having a proximal end and a distal end; and
a debridement tool coupled to the distal end of the shaft, wherein the debridement device is movable between a straight position and a plurality of bent positions and wherein the debridement device automatically returns substantially to the straight position when exposed to heat above a transformation temperature.

40. A method of treating a patient, comprising:
exposing a musculoskeletal lesion;
bending a debridement device from a substantially straight position to one of a plurality of bent positions;
inserting an end of the debridement device into the patient; and
removing the musculoskeletal lesion using the debridement device.

41. The method of claim 40, further comprising:
removing the debridement device from the patient.

42. The method of claim 41, further comprising:
exposing the debridement device to heat above a transformation temperature in order to return the debridement device to a substantially straight position.
